(19) United States
(12) Patent Application Publication
(10) Pub. No.: US 2003/0044801 A1
(43) Pub. Date: Mar. 6, 2003

(54) Array using microspheres

(76) Inventor: Thomas B. Harvey III, Scottsdale, AZ (US)

Correspondence Address: J. Matthew Buchanan, BRINKS HOFER GILSON & LIONE, P.O. Box 10395, Chicago, IL, 60610, US

(21) Appl. No.: 09/946,424

(22) Filed: Sep. 5, 2001

(51) Int. Cl. C12Q 1/68; G01N 33/53; C12M 1/34

(52) U.S. Cl. 435/6; 435/7.1; 435/287.2

(57) ABSTRACT

A microarray containing microspheres is provided. A substrate defines an arrayed pattern of test sites, and one or more microspheres are retained in proximity to each test site by an attachment point disposed on the test site. Each microsphere retained at a particular test site has a common ligand on its surface, which binds a particular analyte. Preferably, different ligands are used at each of the test sites, allowing for the detection of multiple analytes. A method of fabricating a microarray according to the present invention is also provided.

### FIELD OF THE INVENTION

The present invention relates to apparatuses for detecting one or more analytes. More specifically, the invention relates to an apparatus having an arrayed pattern of microspheres that have associated ligands. The ligands are able to interact with the analytes in a chemically specific manner. Further, the invention relates to a method of fabricating such an apparatus.

### BACKGROUND OF THE INVENTION

Microarrays have utility in a variety of applications in which it is desirable to screen a sample for the presence of one or more anlaytes. For example, by using a microarray of polynucleotide probes, a researcher can evaluate a sample for the presence of hybrids to the various probes. In this example, the hybrids, which are the analytes, can comprise transcripts of a particular gene. Using this type of microarray, a researcher can efficiently screen a sample for the presence of transcripts of a plurality of genes, allowing for the construction of a gene expression profile.

Microarray technology takes advantage of the specific biochemical interactions between a particular ligand and a particular analyte. As indicated above, the ligand and analyte can comprise a polynucleotide and its hybrid, respectively. Also, any other type of ligand/analyte set can be used, such as antibodies and antigens.

The ability to use microarrays to screen for numerous analytes is greatly facilitated by the specific arrayed arrangement of the ligands on a substrate. Typically, the ligands are arranged on a substrate such that each ligand has a specific location, usually identified by an X,Y coordinate system. Once an analyte is detected, its identity is determined by resolving the identity of the ligand to which it is bound. This is easily accomplished by querying a database for the identification of the ligand associated with the appropriate set of X,Y coordinates.

While the arrayed arrangement of ligands makes microarrays such useful tools, it also makes them difficult to manufacture. In the conventional fabrication process, large numbers of ligands are systematically bound directly to small defined regions on a substrate in a defined pattern. The use of direct binding of the ligand to a substrate limits the chemistries that can be used to those that allow for the efficient attachment of a large quantity of ligands to a specific substrate.

### SUMMARY OF THE INVENTION

The present invention provides a microarray apparatus that utilizes microspheres. The apparatus of the present invention allows for the efficient screening of a sample for a large number of analytes. In one embodiment, the apparatus comprises a substrate having a test site that can be identified by its position on the substrate, at least one microsphere with an attached ligand that is capable of interacting with one of the analytes in a chemically specific manner, and at least one attachment point disposed on the test site. The attachment point is capable of retaining the microsphere in proximity with the test site, thereby placing the ligand in substantially the same location (i.e., the same X,Y coordinate position) on the substrate as the test site.

The present invention also provides a method of fabricating a microarray apparatus in accordance with the present invention. A preferred method comprises providing a plurality of microspheres, providing a substrate, patterning an array of attachment points onto the substrate, and contacting the microspheres with the substrate such that the attachment points substantially retain the microspheres. Ligands capable of interacting with analytes in a chemically specific manner can be attached to the microspheres before, during, or after contacting the microspheres with the substrate.

### BRIEF DESCRIPTION OF THE DRAWINGS

FIG. 1 is a schematic of a microarray according to the present invention.

FIG. 2 is a schematic of a microarray according to a first embodiment of the present invention and illustrates a relationship between the substrate, attachment points and microspheres.

FIG. 3 is a schematic, partially broken away, of a microarray according to a second embodiment of the present invention.

### DETAILED DESCRIPTION OF THE INVENTION

As used herein, the term “analyte” refers to a molecule or other substance present in a sample being evaluated that is able to bind, in a chemically specific manner, to a ligand used to probe the sample for the analyte. Examples of analytes that could be detected by apparatuses in accordance with the present invention include, but are not limited to, polynucleotides such as mRNA and cDNA, proteins, antigens, sugars, lipids, phospholipids, whole eukaryotic cells, whole prokaryotic cells, chemical species, cell-bound receptors, cytokines, metabolites, and drugs.

As used herein, the term “ligand” refers to a molecule or other substance that is able to bind, in a chemically specific manner, a particular analyte or analytes of interest.

As used herein, the phrase “chemically specific manner” refers to a chemical interaction between two chemical entities, such as a ligand and analyte, that arises due to an affinity one entity has for the other. The chemical interaction constitutes specific binding, as opposed to non-specific binding. Accordingly, the interactions between chemical entities suitable for use in the present invention typically have K_{D} valves ranging from about 10⁻⁶ (typical for specific protein interactions) to 10⁻¹⁴ (typical for antibody/antigen interactions).

As used herein, the term “array” refers to an ordered spatial arrangement of items that allows each item to be identified by its position within the array. Preferably, an array includes identifying indicia, such as an X,Y coordinate system that facilitates such identification of the arrayed items.

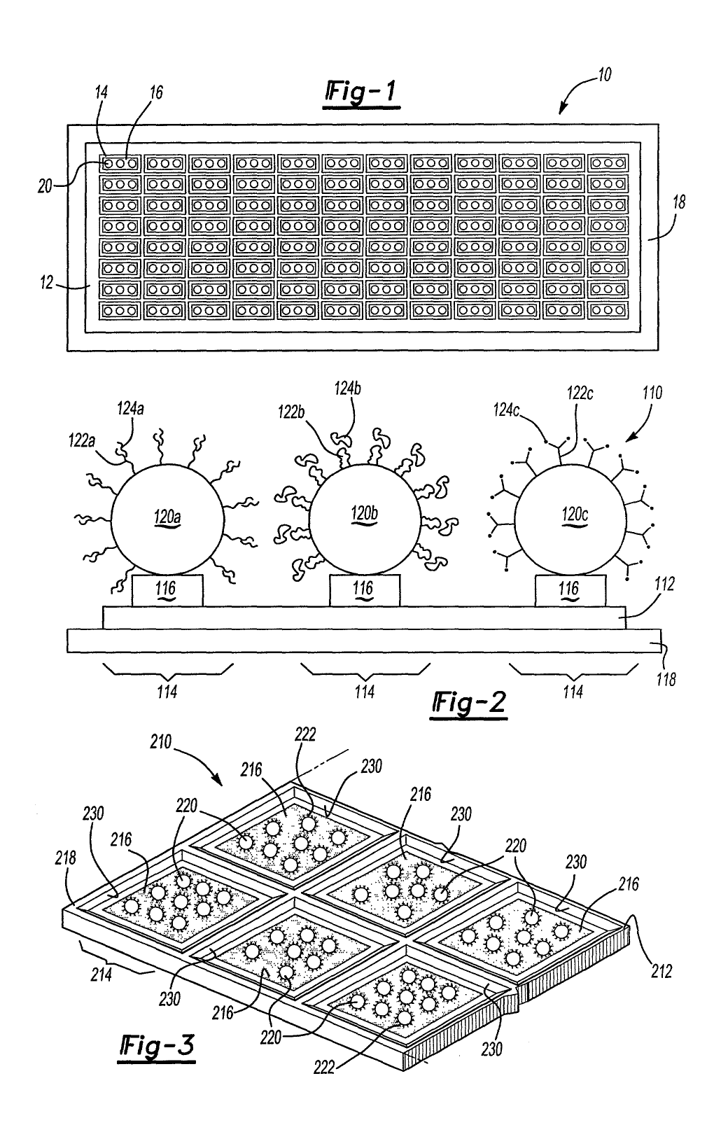

FIG. 1 illustrates a schematic of an apparatus in accordance with the present invention. The apparatus, generally indicated in the figure at reference 10, includes a substrate 12, at least one test site 14, at least one attachment point 16 disposed on the test site 14, an optional support member 18 underlying the substrate 12, and at least one microsphere 20 bound to the attachment point 16 and in proximity with the test site 14.

The apparatus 10 is useful in the screening of a sample for the presence of numerous analytes. The apparatus 10 provides a platform onto which many different analyte assays can be based. By providing an attachment point 16 at which microspheres 20 can readily bind, the apparatus 10 allows for assay customization by binding a microsphere 20 with a ligand to an analyte of interest to the attachment point 16. A plurality of microspheres 20 with a variety of ligands can also be used. An investigator need not develop chemistry to bind the ligand directly to the substrate.

The substrate 12 provides a surface onto which the attachment points 14 can be disposed. The substrate 12 bears one or more regions, referred to as test sites 14, onto which the attachment points 14 are disposed. Preferably, as illustrated in FIG. 1, the test sites 14 are arranged into an array pattern on the substrate 12. The array pattern is conventional in analyte detection and provides an efficient mechanism for indexing a plurality of ligands. The substrate 12 can include a single test site 14 or a plurality of test sites 14. The number of test sites available will depend on the available surface area on the substrate. Preferably, the substrate has a surface area of between 0.01 μm² and 5 cm², and contains between 1 and 1×10⁸ test sites. Particularly preferable, the substrate has a surface area of between 1 mm² and 1 cm², and contains between 1×10³ and 1×10⁶ test sites. While any suitable number and arrangement of test sites 14 can be utilized, examples of other preferred quantities and arrangements include those commonly used in conventional analyte detection methods and apparatuses, such as grids of 96, 384 and 1536 test sites. Also preferable, the number and pattern of test sites 14 correlates with the form of the support member 18 (if present), which will be more fully developed below.

The test sites 14 are preferably arranged on the substrate 12 such that binding interactions between microspheres 20 and attachment points 16, as well as between ligands on the microspheres 20 and appropriate analytes, at one test site 14 are not hindered by those at another test site 14. While the exact spacing between test sites 14 will depend on numerous factors, including size of the substrate 12 and microspheres 20, the test sites 14 are preferably arranged on the substrate 12 such that they are separated by a distance of from about 0.05 μm to about 0.5 mm. Particularly preferable, the test sites are separated by a distance of between about 0.10 μm and 10 mm. Also, as illustrated in FIG. 1, the test sites 14 are preferably positioned on the substrate 12 in a regular pattern with a uniform spacing between all adjacent test sites 14.

The substrate 12 preferably defines the test sites 14 and provides support for the attachment points 16. Alternatively, the substrate can also define the attachment points, as will be developed more fully below (see Second Preferred Embodiment). Any suitable backing that allows disposition of the attachment points 16 in the desired pattern can be utilized for the substrate 12. Suitable substrates include solid materials such as glass, silicon, silicon nitride, plastic, rubber, fabric, ceramic, a printed circuit board, or combinations thereof. Also, gel materials such as agarose and polyacrylamide can be used. Preferred materials include plastics and glass.

The test sites 14 represent regions of the substrate 12 onto which the attachment points 16 are disposed. The test sites 14 can be a region defined by a series of X,Y coordinates, a bottom surface of a well, or any other identifiable region on the substrate.

The attachment points 16 are disposed on the test sites 14 of the substrate 12, and are thus arranged in an array pattern that is determined by the array pattern of the test sites 14. Preferably, as illustrated in FIG. 1, a single attachment point 16 is disposed on each individual test site 14 of the substrate 12.

The attachment points 16 are capable of retaining one or more microspheres 20 in proximity to the test site 14. Preferably, the attachment points 16 are functional moieties that are able to chemically bind to the microsphere 20 in a manner that retains the microsphere near the test site 14. For example, a binding agent having first and second binding regions can be used. This first binding region is able to bind the substrate, and the second binding region is able to bind to the microsphere. Alternatively, any other suitable binding agent that is able to couple the microsphere to the substrate can be used. Also alternatively, any adhesive that is able to retain the microsphere can be utilized. In this embodiment, an adhesive can be disposed on the test site. Suitable adhesives include, for example, epoxy adhesives. Other alternatives for the attachment point include areas of the substrate that have a higher affinity for the microsphere than the surrounding areas. For example, the test sites of the substrate could contain nickel ions, which would enable the test site to bind microspheres bearing polyhistidine tags. In this embodiment, areas of the substrate that surround these areas of high affinity would be free of nickel ions, or have relatively lower concentrations of nickel ions.

A support member 18 can optionally be disposed under the substrate 12. The support member 18 provides additional support to the apparatus 10, and may facilitate handling of the apparatus, both by individuals and testing equipment. The support member 18 can be the same material as the substrate or different. Thus, suitable materials for the support member 18 include glass, silicon, silicon nitride, plastic, rubber, fabric, ceramic, a printed circuit board, or combinations thereof.

Preferably, the support member 18 defines a form that facilitates the use of the apparatus 10 in analyte detection assays. Suitable forms for the support member 18 include those conventional in the art. For example, the support member 18 can be a microtiler plate, known to those skilled in the art. In this embodiment, the plate preferably defines one or more wells, and one or more test sites are disposed on the bottom of the individual wells. Other examples of suitable support members include microscopic slides and tissue flasks.

The microsphere 20 provides a solid support for appropriate ligands. Microspheres of varying size and shape have previously been used in a variety of scientific applications, including chemical synthesis and separation and/or purification methods. The preparation and use of microspheres to bind a ligand is known to those skilled in the art. Indeed, microspheres of various sizes and materials are readily available from several commercial sources.

Essentially, any solid support that is able to bind the ligand(s) of interest as well as the substrate, either directly or through a linker group, can be utilized as the microsphere. Examples of suitable materials for the microspheres include polyacrylamide, agarose, polyethylene glycol, cellulose, sol gel, glass, nylon polypyrrole, polythiphene, polyaniline, polypyridine, polycarbazole, polyphenylene, poly(phenylenvinylene), polyfluorene, polypropylene, polystyrene, polyindole, polyacrylates, polymethacrylates, and polycarbonates, or their derivatives, copolymers, or combinations thereof.

Typical microsphere are spherical in shape and range in size from approximately 0.2 μm to 100 μm in diameter. Preferred microspheres range in size from about 0.5 μm to 10 μm. Particularly preferable, the microspheres are about 1 μm in diameter. Due to their ready availability, microspheres that are substantially spherical are preferred for use in the present invention. The size of the microspheres can be optimized based on whether it is desirable to have a single microsphere or a plurality of microspheres disposed on each attachment point, as will be developed more fully below.

### DESCRIPTION OF PREFERRED EMBODIMENTS OF THE INVENTION

The following description of two preferred embodiments provides a detailed description of the invention. The embodiments discussed herein are exemplary in nature, and are not intended to limit the scope of the invention in any manner.

### First Preferred Embodiment

FIG. 2 illustrates an apparatus 110 for detecting one or more analytes according to a first preferred embodiment of the present invention. Like reference numbers in FIG. 2 refer to similar features and/or components illustrated in FIG. 1.

In this embodiment, a single microsphere 120 is retained by each attachment point 116. As such, the microspheres 120 are arranged in the same array pattern as the attachment points 116. Likewise, each test site 114 has a single associated attachment point 116. Thus, the microspheres 120 are arranged in the same array pattern as the test sites 114.

Preferably, each microsphere 120 bears a unique ligand relative to the other microspheres. As shown in the figure, microsphere 120a bears a polynucleotide ligand 122a, which binds analyte 124a, a complimentary polynucelotide. Microsphere 120b bears ligand 122b, a globular protein such as the extracellular portion of a receptor, which binds analyte 124b. Similarly, microsphere 120c bears ligand 122c, an antibody, which binds analyte 124c, an antigen.

It should be noted that numerous different ligands and types of ligands can be utilized in a single apparatus. For example, an entire library of nucleic acid probes (e.g., a single probe to thousands of different probes) can be used, with each microsphere bearing a unique ligand relative to every other microsphere. It will be appreciated that the ligand 118 can comprise any molecule capable of binding one or more of the analytes 124 of interest with the desired specificity. Examples of suitable ligands include polynucleotides, polypeptides, proteins, carbohydrates, monoclonal antibodies, polyclonal antibodies, polyclonal antisera, members of a natural products library, members of a peptide library, members of a phage display library, members of a combinatorial library, or fragments or combinations thereof. The ligand can also comprise a metal chelating agent, which would allow for assaying of metals as analytes. Various metal chelating agents are known to those skilled in the art, and the choice of agent can be made based on the metal(s) of interest. Preferably, as shown in FIG. 2, multiple copies of the same ligand are distributed on the microsphere 120.

The ligand 112 can be directly linked to the microsphere 120, or a linker group can be used. The linker group can be any entity that binds to the microsphere and binds to the ligand. Preferably, the linker group binds at least the ligand in a specific manner. For example, the linker group can comprise streptavidin, which would bind to biotinylated ligands, or vice versa. The use of streptavidin/biotin to link chemical entities is well known in the art and will not be described in detail herein. If a larger clearance is desired between the microsphere and the ligand, a polymeric material can be used as the linker group. Examples of suitable polymeric materials for use as the linker group include polyethylene glycols, and oligomers of various other polymeric materials.

### Second Preferred Embodiment

FIG. 3 illustrates an apparatus 210 for detecting one or more analytes according to a second preferred embodiment of the present invention. The second preferred embodiment is similar to the first preferred embodiment, except as described below. Accordingly, like reference numbers in FIG. 3 refer to similar features and/or components in FIGS. 1 and 2.

In this embodiment, several microspheres 220 are retained by each attachment point 216. As a result, the microspheres 220 are arranged in the array pattern of the test sites. In contrast to the first preferred embodiment, however, several microspheres 220 are in proximity with each test site 214. Preferably, all microspheres 220 at each test site 214 bear the same ligand 222. That is, it is preferred that all microspheres 220 at a single test site 214 have a common ligand 222. Also preferable, the ligand 222 associated with the microspheres 220 at one test site 214 are unique from those associated with the microspheres 220 associated with at least one other test site 214. Particularly preferable, the ligand 222 associated with the microspheres 220 at one test site 214 are unique as compared to those associated with all other test sites 214 in the apparatus 210.

The figure also illustrates the use of a plate as the support member 218. In this embodiment, the support member 218 also serves as the substrate 212. The plate 218 defines a series of wells 230 arranged in an arrayed pattern. Each well 230 contains a portion of the substrate 212, an attachment point 214, and a plurality of microspheres 220. Each test site 214 preferably comprises the bottom of a single well 230. As described above, the attachment point 216 can comprise the substrate 212, i.e., the microspheres 220 bind directly to the substrate 212, or the attachment point 216 can comprise another entity, such as a binding agent or layer of adhesive disposed on the substrate 212.

### METHODS OF FABRICATING AN APPRATUS FOR DETECTING ANALYTES

The present invention also provides methods of fabricating an apparatus for detecting analytes. A preferred method comprises providing a plurality of microspheres, providing a substrate, patterning an array of attachment points onto the substrate, and contacting the microspheres with the substrate such that the attachment points substantially retain the microspheres.

The methods of fabricating may further comprise removing any microspheres not retained by the attachment points. This step is preferably accomplished by rinsing the substrate with a fluid. Preferably, the fluid is chosen so as not to interfere with the interaction between the attachment points and the retained microspheres.

The methods may also further comprise attaching a series of ligands to the plurality of microspheres. Preferably, each ligand in the series is capable of interacting with one of the analytes to be detected. Also preferable, and as described above, this step is performed such that the microspheres retained by or to be retained at a particular test site bear a common ligand that is unique as compared to the ligands on microspheres retained at at least one other test site. It should be noted that attaching a series of ligands to the plurality of microspheres can be performed either prior to, during, or subsequent to contacting the microspheres with the substrate.

The patterning an array of attachment points onto the substrate can be accomplished by any suitable technique. Preferably, the technique chosen is optimized for the type of attachment point used. For example, if an adhesive is used as the attachment point, a masking or silk-screen printing application of the adhesive to the substrate will suffice. If other types of attachment points are used, other techniques may be desirable. Examples of suitable techniques for this step includes photolithography, silk-screen printing, masking, micro-contact and direct contact printing techniques. For general information on various microarray printing techniques, including direct contact printing, see U.S. Pat. No. 6,101,946 to Martinsky, for a MICROARRAY PRINTING DEVICE INCLUDING PRINTING PINS WITH FLAT TIPS AND EXTERIOR CHANNEL AND METHOD OF MANUFACTURE.

The foregoing disclosure is the best mode devised by the inventors for practicing the invention. It is apparent, however, that several variations in microarrays in accordance with the present invention may be conceivable by one skilled in the art. Inasmuch as the foregoing disclosure is intended to enable one skilled in the pertinent art to practice the instant invention, it should not be construed to be limited thereby, but should be construed to include such aforementioned variations. As such, the present invention should be limited only by the spirit and scope of the following claims.

1. An apparatus for detecting one or more analytes is a sample, comprising:
a substrate having a test site identifiable by its position on the substrate;
a microsphere in proximity with the test site and attached to a ligand that specifically binds with one of said analytes; and
at least one attachment point disposed on the test site, which retains the microsphere in proximity to the test site.

2. An apparatus in accordance with claim 1, wherein the substrate has a plurality of test sites.

3. An apparatus in accordance with claim 2, wherein a single attachment point is disposed on each test site.

4. An apparatus in accordance with claim 3, wherein each attachment point retains a single microsphere.

5. An apparatus in accordance with claim 3, wherein each attachment point retains a plurality of microspheres.

6. An apparatus in accordance with claim 5, wherein all microspheres at a single test site have a common ligand.

7. An apparatus in accordance with claim 2, wherein the microsphere in proximity with a particular test site has a unique ligand relative to ligands attached to microspheres in proximity with at least one other test site.

8. An apparatus in accordance with claim 1, wherein the microsphere comprises polyacrylamide, agarose, polyethylene glycol, cellulose, sol gel, glass, nylon, polypyrrole, polythiphene, polyaniline, polypyridine, polycarbazole, polyphenylene, poly(phenylenvinylene), polyfluorene, polypropylene, polystyrene, polyindole, a polyacrylate, a polymethacrylate, or a polycarbonate.

9. An apparatus in accordance with claim 1, wherein the ligand comprises a polynucleotide, a polypeptide, a protein, a carbohydrate, a monoclonal antibody, polyclonal antibodies, a polyclonal antiserum, a member of a natural products library, a member of a peptide library, a member of a phage display library, a member of a combinatorial library, a metal chelating agent, or fragments or combinations thereof.

10. An apparatus in accordance with claim 1, wherein the attachment point comprises a binding agent having first and second binding regions, the first binding region being able to bind the substrate and the second binding region being able to bind the microsphere.

11. An apparatus in accordance with claim 1, wherein the attachment point comprises the substrate.

12. An apparatus in accordance with claim 1, wherein the attachment point comprises an adhesive.

13. An apparatus in accordance with claim 1, wherein the attachment point comprises an area of the substrate having an increased affinity for the microsphere.

14. An apparatus in accordance with claim 1, further comprising a support member underlying the substrate.

15. An apparatus in accordance with claim 14, wherein the support member comprises glass, silicon, silicon nitride, plastic, rubber, fabric, ceramic, a printed circuit board, or a combination thereof.

16. An apparatus in accordance with claim 14, wherein the support member comprises a plate defining one or more wells.

17. An apparatus in accordance with claim 14, wherein the support member comprises the substrate.

18. An apparatus in accordance with claim 1, further comprising a linker group disposed on the microsphere and attached to the ligand.

19. An apparatus in accordance with claim 18, wherein the linker group comprises streptavidin and the ligand is biotinylated.

20. An apparatus in accordance with claim 18, wherein the ligand comprises streptavidin and the linker group is biotynylated.

21. A method of fabricating an apparatus for detecting a plurality of analytes, comprising:
providing a plurality of microspheres;
providing a substrate defining an array of test sites;
patterning an array of attachment points onto the array of test sites, each attachment point being able to retain at least one of the microspheres in proximity to a test site;
contacting the microspheres with the substrate such that the attachment points substantially retain the microspheres in proximity to the test sites.

22. A method in accordance with claim 21, further comprising removing any microspheres not retained by the attachment points.

23. A method in accordance with claim 22, wherein removing any microspheres not retained by the attachment points comprises rinsing the substrate with a fluid.

24. A method in accordance with claim 21, further comprising attaching a series of ligands to the plurality of microspheres, wherein each ligand specifically binds one of said analytes.

25. A method in accordance with claim 24, wherein attaching a series of ligands to the plurality of microspheres is performed prior to contacting the microspheres with the substrate.

26. A method in accordance with claim 21, wherein providing a plurality of microspheres comprises providing a plurality of microspheres in which each microsphere has an attached ligand capable of interacting with one of said analytes in a chemically specific manner.

27. A method in accordance with claim 21, wherein patterning an array of attachment points onto the array of test sites is accomplished by photolithography, silk-screen printing, masking, or micro-contact printing techniques.

28. An apparatus for detecting one or more analytes, comprising:
a substrate defining a plurality of test sites arranged in an array pattern, each test site being identifiable by its position on the substrate;
a plurality of microspheres in proximity with each test site, each microsphere having an attached ligand capable of interacting with one of said analytes in a chemically specific manner and all microspheres in proximity with a single test site bearing a common ligand;
at least one attachment point disposed on the test site, the attachment point being capable of retaining at least one of the microspheres in proximity to the test site.
